(19) United States
(12) Patent Application Publication
(10) Pub. No.: US 2022/0409872 A1
(43) Pub. Date: Dec. 29, 2022

(54) CHEM-O-PORT COVER

(71) Applicants: Brittany GRINER, Macon, GA (US); Renee GRINER, Columbus, GA (US)

(72) Inventors: Brittany GRINER, Macon, GA (US); Renee GRINER, Columbus, GA (US)

(21) Appl. No.: 17/357,580

(22) Filed: Jun. 24, 2021

(51) Int. Cl. A61M 35/00; A61M 39/02

(52) U.S. Cl. CPC A61M 35/10; A61M 39/0247; A61M 2039/0276; A61M 2039/0288

(57) ABSTRACT

A chemo-o-port cover includes flexible and stretchable material sized to cover the skin surface immediately above the implanted port and a centered ring structure secured on its surface to hold numbing cream. The thickness of the cover permits the cover to provide a barrier and movement. The height and diameter of the ring defines a volume sufficient to contain an amount of numbing cream to reduce or eliminate the pain associated with injecting the chemotherapy medicament into the implanted port structure. The stretchable material also contains adhesive regions to secure the cover to the skin of the patient and keep the cover in place for long periods of time. The adhesive permits repeated use of the cover—removal and reapplication.

### FIELD OF THE INVENTION

The invention relates generally to minimizing the pain associated with the administration of medicaments through needle into an implanted chemo-o-port system.

### BACKGROUND OF THE INVENTION

Chemotherapy can include the use of chemo-port to facilitate prolonged treatment regimens. The port is surgically inserted and threaded into a large vein typically above the right side of the heart. The port becomes the access point where the chemotherapy is administered to the body. A large needle is inserted into the port that administers the drug and it can be a source of great pain for patients who are already suffering. One of the ways of addressing this pain is through the administration of a numbing cream over the port before the administration of the needle into the port. The standard medical practice throughout the industry is to put the numbing cream over the port and then put a piece of plastic wrap or gauze over the cream to try to keep the cream in place. This approach presents some difficulties. For example, the plastic wrap fails to hold the cream in a concentrated manner over the port which lessens the numbing effect. Also, the tapping of the plastic wrap over the port to hold it in place causes irritation of the very sensitive skin of the patient.

Recently, some cancer centers have been using a lidocaine spray to spray over the port before needle insertion to address the problem. However, this approach can cause the patients pain and even change the color of the skin.

In addition, as described in an article attributed to the University of Michigan Health Systems, a solution to this issue is to apply the numbing cream one hour before the patient's chemotherapy session and do so by squeezing out an amount about the size of a quarter onto the port area. Then they recommend covering the cream to increase the numbing effect by either using a non-stick dressing or plastic wraps, such as Saran Wrap®, Press ‘N’ Seal®, or a plastic sandwich bag cut open and securing it with tape if needed. They do not recommend using gauze or Band-Aids on cream as they will absorb the cream. Other articles that discuss solutions to this issue recommend using a Tegaderm™ dressing which is essentially a clear plastic dressing with Band-Aid-like sticky surfaces around the edges.

Further, an article describing the work of chemotherapy nurses at Rutgers Cancer Institute of New Jersey Hamilton suggests placing a plastic cap from a medicine bottle, concave side down, over the cream, then taping the bottle cap down with first-aid tape.

While these efforts may represent partial solutions to this pain issue, there remains a need to localize numbing cream onto the port for a maximum numbing effect that allows standardization and repeatability. Also, adhesion of a device on the skin above the port has address the irritation problem caused by adhesive tape from skin of the patient made sensitive from the presence of the port. These are the same issues that are faced with using the Tegaderm™ dressing or something similar to it.

### SUMMARY OF THE INVENTION

The invention involves a chemo-port cover with a centered ring structure and an adhesive region surrounding the centered ring structure. The ring is sized to cover completely the area of skin immediate to the implanted port and to contain sufficient amounts of the numbing cream to provide the desired effect over the time required for injection. The adhesive is located on the cover to secure it at the implant site and to allow its removal and reattachment. The shape of the cover can vary depending on the implant site location. The shape can also provide for tabs so that it can be removed with minimal effort and minimize tugging skin that could cause pain or irritation.

With our invention, the patient can wear this cover with the center ring without disrupting their normal day-to-day lives without worries about daily items getting caught on this port because the cover has a sleek surface that covers the port and protects it from outside protrusions. This invention is also something that these patients can use multiple times which saves them money and time.

### BRIEF DESCRIPTION OF THE DRAWINGS

FIG. 1 shows a perspective view of a protective cover with the centered ring structure according to the invention.

FIG. 2 shows a side view of the protective cover with the centered ring structure.

FIG. 3 shows a bottom view of the cover with the centered ring structure.

FIG. 4 shows a tope view of the protective cover as it would appear when positioned over the implanted port.

FIG. 5 shows protective cover and ring structure of the invention.

### DETAILED DESCRIPTION OF THE INVENTION

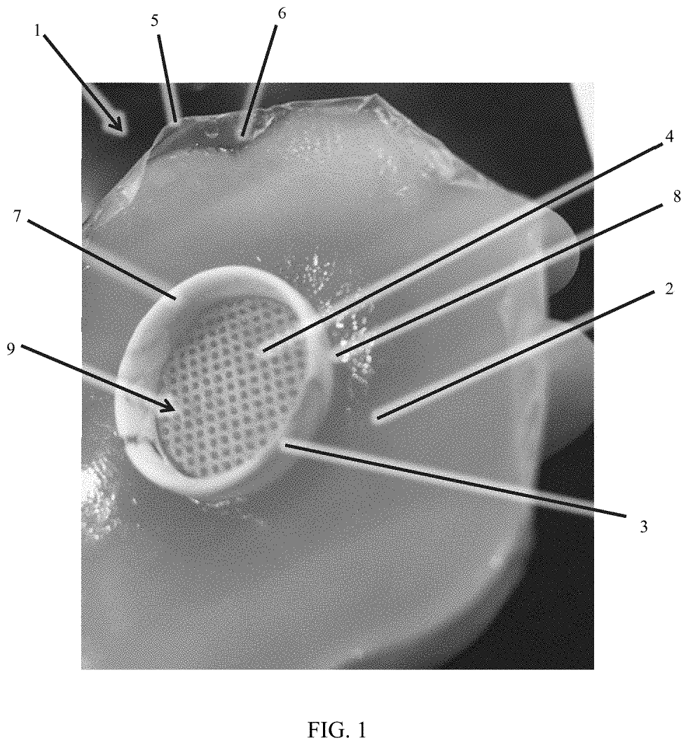

Referencing FIG. 1, shown is an angled bottom view of the protective cover (1) having a dressing portion (2) and an attached centered ring structure (3). An optional centrally placed polymeric grid structure (4) is centered on the protective cover. When present, the grid is either surrounded by the ring structure or is in direct contact with the ring structure. The dressing portion (2) has an edge (5), a boundary, and an adhesive portion (6) coextensive with the edge that causes the cover to adhere to the skin of the patient. The boundary can define a variety of shapes, e.g. oval, oblong, etc., that can include one of more tabs. The cover (1) is attached to the patient above where the implanted chemo port (not shown) has been placed so that the surface is covered. The adhesive portion (6) provides either a continuous or continual seal between the skin of the patient and the cover (1). The dressing portion is transparent and flexible. The dressing is flexible allowing the patient to move about without the seal loosing contact. The adhesive keeps the cover (1) centered over the implanted chemo port. The adhesive is of a type that permits repeated removal and attachment of the cover (1). The attached ring structure (3) has an outer lip (7) and an inner edge (8) that is in contact with the dressing portion. The contact with the dressing of grid structure affords permanence. It is adfixed. The ring structure (3) and the dressing portion (2) define a concave structure, a cavity, (9) for holding a volume of numbing cream. The volume of the cavity (9) is sufficient to contain an amount of numbing cream to avoid the pain associated with the injection with the needles associated with the administration of medicaments or tissue sampling. The ring structure can be breathable structure and be composed of clinically safe materials, e.g. silicone.

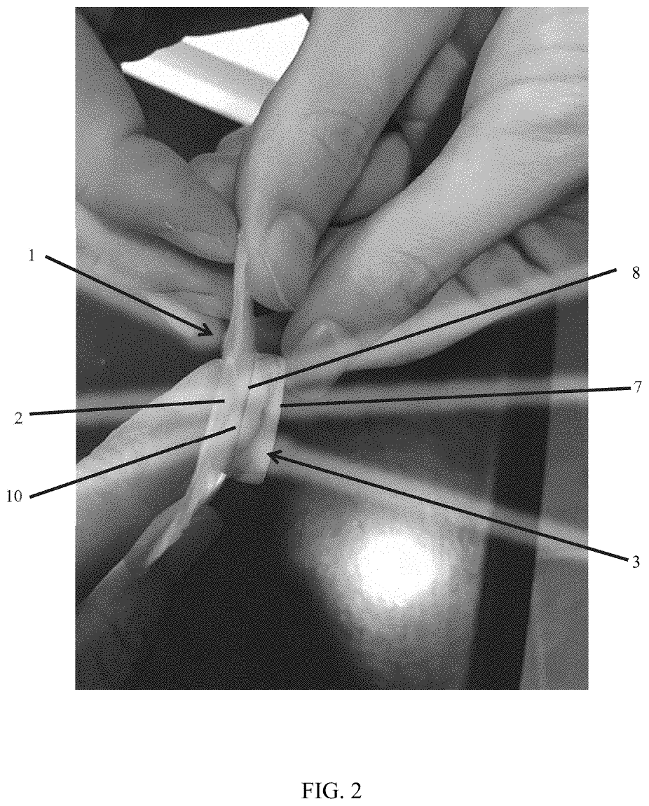

Referencing FIG. 2, the photograph shows a side view of the protective cover (1) where the ring structure (3) is in contact with the dressing portion (2). The area of contact (10) can include adhesive or like material. The adhesive fixes the ring structure to the dressing portion (2) or plastic grid. Also shown in the photograph is the outer lip (7) of the ring structure and an inner edge (8).

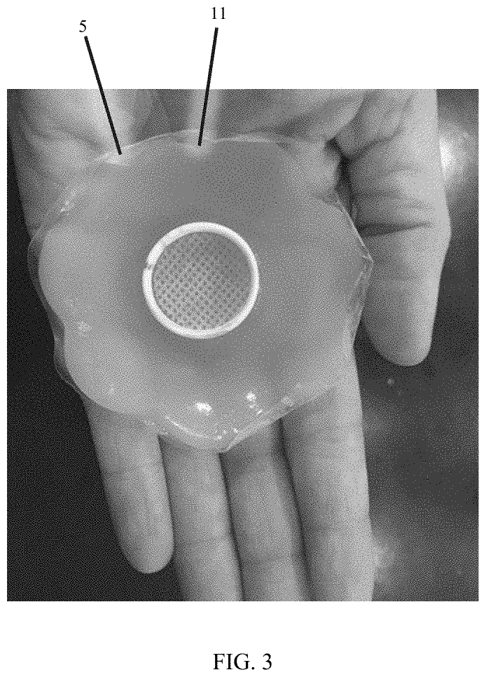

Referencing FIG. 3, shown is the bottom view of invention shown in FIG. 1 where the tabs (11) that form the boundary (5).

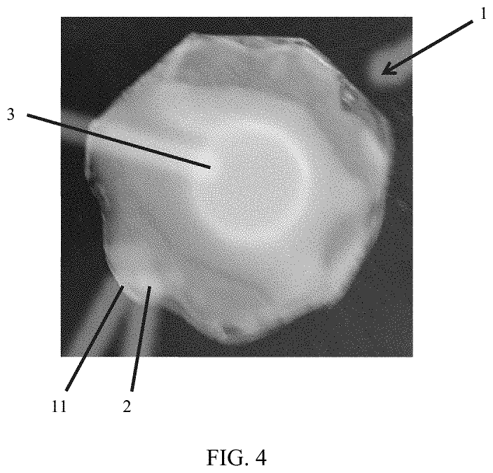

Referencing FIG. 4, the photograph shows a top view of the protective cover (1) of the protective cover (1) which is transparent. Because of the transparency, the ring structure can be seen along with the tab structures of the dressing portion (11),

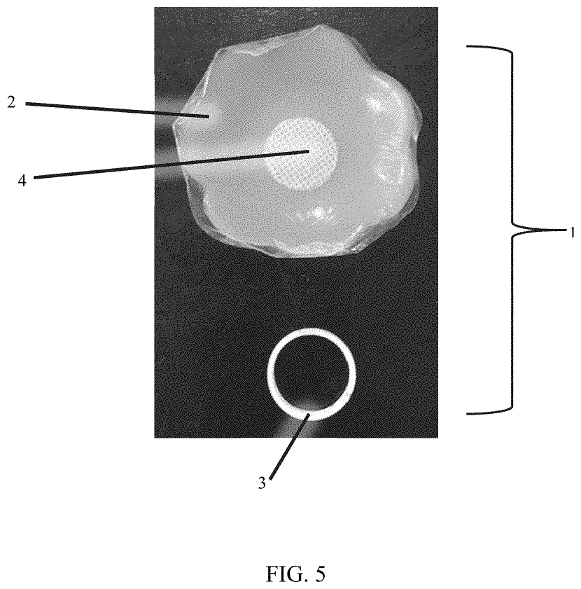

Referencing FIG. 5, the photograph shows component parts of the protective cover components, i.e., the dressing portion (2) with the polymeric grid material (4) and ring structure (3). The optional polymeric grid (4) is either an integral part of the dressing portion (2) or is affixed to it.

### Operation

The invention is directed to an improved chemo port cover where a ring structure is employed to concentrate the numbing cream at the injection site, the skin of the subject above the implanted port. Implanted ports are usually placed about an inch below the skin surface. The implanted port is typically a flexible tube place into the vein in the chest. The port makes it easier to give intravenous medications and fluids, take blood samples and give medications continuously over several days. Waterproof injection port covers are known, e.g. U.S. Pat. No. 6,222,090. Adhesives that permit multiple use of medical equipment are well known, e.g. U.S. Pat. No. 9,017,771, as are breathable ring structures. The dressing material can be akin to adhesive clothing material, U.S. Pat. No. 6,897,935.

The chemo-o-port cover consists of dressing portion of stretchable material sized to cover at least the surface area of an implanted port with a centered ring structure secured on the cover. The ring defines volume that is sufficient to hold the numbing cream medicament, e.g. lidocaine, in amounts that numb the skin and avoid or lessen the pain associated with needle injections. The region surrounding the ring, particularly the region between the edge of the dressing portion and the ring, has an adhesive material sufficient to secure the cover to the skin.

The ring dimensions can vary. However, for accurate adult port size: 1) the ring size is typically 11 (20.6 mm): width 6 mm wide/thickness: 2 mm, 2) breathable design and 3) composed of premium, silicone. The rings are all made of pure medical grade silicone. They're comfortable, smooth, non-conductive and definitely safe even for sensitive skin, not to be damaged from heavy work or strenuous exercise like electrical work, swimming, weight lifting, etc.

A method of administering a numbing cream on a subject undergoing chemotherapy at a chemo-o-port site includes providing a chemo-port cover with a centered ring, introducing numbing cream, e.g. lidocaine, into a cavity formed by the ring and the cover structures; and positioning the cover on the subject so that the cream contained within ring structure is in contact with the surface of the skin of the subject immediate to the port and the adhesive region is in contact with the subject securing the cover. The cover is allowed to remain on the subject for at least one hour or for a time sufficient to numb the skin. After the skin is numbed, the needle can be inserted. The subsequent act can then take place, e.g. injecting a medicament into the port.

The components of the protective cover port including the ring can be assembled in a kit form to facilitate handling. The kit can include the assembled cover or the component parts. The component parts can include various sizes of rings and/or dressing sizes. The kit can also include the numbing cream and/or instructions for use.

While the invention has been described with an emphasis on preferred embodiments, it will be obvious that variations of the preferred embodiments may be used, and that it is intended that this invention may be practiced otherwise than as specifically designed herein. Accordingly, this invention includes all modifications encompassed within the spirit and scope of the invention as defined by the following claims.

1. A chemo-o-port cover comprising cover structure of stretchable material sized to cover at least the surface area of an implanted port wherein the cover contains a centered ring structure secured on the cover and capable of holding a numbing cream medicament and a region surrounding the ring with an adhesive material sufficient to secure the cover to the skin.

2. A method of administering a numbing cream on a subject undergoing chemotherapy at a chemo-o-port site comprising,
providing a chemo-port cover described in claim 1;
introducing numbing cream into a cavity formed by the ring and the cover structures; and
positioning the cover on the subject so that the cream contained within ring structure is in contact with the surface of the skin of the subject immediate to the port and the adhesive region is in contact with the subject securing the cover.

3. The method of claim 2 further comprising maintaining the secured cover on the subject for a time sufficient to numb the skin prior to injecting a medicament into the port.

4. In a kit for administering a numbing cream on a subject undergoing chemotherapy at a chemo-o-port at a chemo-o-port site wherein the improvement comprises the chemo-o-port cover of claim 1.
